(19) United States
(12) Patent Application Publication
(10) Pub. No.: US 2017/0351836 A1
(43) Pub. Date: Dec. 7, 2017

(54) METHOD AND SYSTEM FOR ESTIMATING FRACTIONAL FAT CONTENT OF AN OBJECT

(71) Applicant: ENDRA, INC., Ann Arbor, MI (US)

(72) Inventors: Michael M. Thornton, London (CA); Jang Hwan Cho, Ann Arbor, MI (US); Aghapi G. Mordovanakis, Ann Arbor, MI (US)

(21) Appl. No.: 15/614,125

(22) Filed: Jun. 5, 2017

Related U.S. Application Data
(60) Provisional application No. 62/345,814, filed on Jun. 5, 2016.

(51) Int. Cl. G06F 19/00; A61B 5/00; A61B 8/08

(52) U.S. Cl. CPC G06F 19/3431; A61B 8/085; A61B 5/4244; A61B 5/4872

(57) ABSTRACT

A method for estimating fractional fat content of an object of interest is described. The method comprises obtaining thermoacoustic data of a region of interest containing an object of interest and a reference, and estimating fractional fat content of the object of interest using the thermoacoustic data and at least one parameter of the reference.

### CROSS-REFERENCE TO RELATED APPLICATIONS

This application claims the benefit of U.S. Provisional Patent Application No.

62/345,814, filed on Jun. 5, 2016, the entirety of which is incorporated herein by reference.

### FIELD

This application relates to a method and system for estimating fractional fat content of an object.

### BACKGROUND

Hepatic steatosis, also known as a fatty liver disease, is a condition where hepatocytes suffer from abnormal intracellular accumulation of fat, mostly in the form of triglycerides (TG). The two main types of hepatic steatosis are alcoholic liver disease (ALD) and non-alcoholic fatty liver disease (NAFLD). NAFLD is the most common cause of chronic liver disease in the United States. Hepatic steatosis can lead to progressive hepatic disease and is a risk factor for cardiovascular disease and diabetes. Liver biopsy with histologic analysis is commonly used for diagnosing and grading a fatty liver. However, due to the invasive nature of liver biopsy with histologic analysis and limitations such as lack of representation of the entire liver, non-invasive assessments based on cross-sectional imaging are being investigated.

Ultrasound imaging has been used for evaluating hepatic steatosis. Ultrasound imaging uses sound waves with frequencies higher than those audible to humans (>20 000 Hz). These sound waves are pulsed into tissue using a probe. The sound waves echo off the tissue. Different tissues reflect difference degrees of sound. These echoes are analyzed through signal processing and are further processed using clinical ultrasound reconstruction algorithms to reconstruct ultrasound images for presentation and interpretation by an operator. Many different types of images can be reconstructed using ultrasound imaging. One such type is a B-mode image which displays the acoustic impedance of a two-dimensional cross-section of tissue. Ultrasound imaging, however, suffers from poor repeatability and reproducibility in evaluating hepatic steatosis.

Unenhanced computed tomography (CT) has been used for evaluating hepatic steatosis. Using unenhanced CT, fatty liver can be diagnosed based on its attenuation value and relative relationship with the spleen and blood. However, the sensitivity of unenhanced CT is limited.

Magnetic resonance imaging (MRI) is currently the most accurate and precise non-invasive imaging modality for diagnosing and quantifying hepatic steatosis. MRI data can be processed to estimate proton density fat fraction (PDFF) as a measure of fractional fat content. However, MRI is expensive.

Although techniques for detecting and grading hepatic steatosis have been considered, improvements are desired. It is therefore an object at least to provide a novel a method and system for estimating fractional fat content of an object using thermoacoustic imaging.

### SUMMARY

Accordingly, in one aspect there is provided a method for estimating fractional fat content of an object of interest, the method comprising obtaining thermoacoustic data of a region of interest containing an object of interest and at least one reference, and estimating fractional fat content of the object of interest using the thermoacoustic data and at least one parameter of the reference.

In an embodiment, the at least one parameter is an absorption coefficient of the at least one reference. The method may comprise estimating an absorption coefficient of the object of interest using the absorption coefficient of the at least one reference.

In an embodiment, the method may further comprise calculating a metric at the boundary using the thermoacoustic data and estimating an absorption coefficient of the object of interest using the metric.

In an embodiment, the method may further comprise grading the object of interest using the estimated fractional fat content.

In an embodiment, the method may further comprise obtaining ultrasound image data of the region of interest, and registering coordinate frames of the thermoacoustic data and ultrasound image data. The region of interest is located using the obtained ultrasound image data. The at least one reference is identified using the obtained ultrasound image data.

According to another aspect there is provided a method for grading an object of interest, the method comprising obtaining thermoacoustic data of a region of interest containing an object of interest and at least one reference, estimating fractional fat content of the object of interest using the thermoacoustic data and at least one parameter of the at least one reference, and grading the object of interest using the estimated fractional fat content.

In an embodiment, the object of interest is a liver and the grading comprises grading hepatic steatosis. The at least one reference is at least one of a blood vessel within the liver and a kidney adjacent to the liver.

According to another aspect there is provided a system comprising a thermoacoustic imaging system, an ultrasound imaging system, and a processing unit configured to process ultrasound image data received from the ultrasound imaging system to generate and display an ultrasound image of a region of interest containing an object of interest and at least one reference, and process thermoacoustic data of the region of interest containing the object of interest and the at least one reference to estimate fractional fat content of the object of interest using at least one parameter of the at least one reference.

According to another aspect there is provided a non-transitory computer-readable medium having stored thereon a computer program comprising computer program code executable by a computer to perform a method comprising obtaining thermoacoustic data of a region of interest containing an object of interest and at least one reference, and estimating fractional fat content of the object of interest using the thermoacoustic data and at least one parameter of the at least one reference.

### BRIEF DESCRIPTION OF THE DRAWINGS

Embodiments will now be described more fully with reference to the accompanying drawings in which:

FIG. 1 is a schematic view of an imaging system in accordance with the subject application;

FIG. 2 is a graph showing exemplary bipolar signals obtained by the imaging system of FIG. 1;

FIG. 3 is a flowchart of a method for grading an object of interest based on the fractional fat content thereof;

FIG. 4 is an exemplary region of interest containing an object of interest and a reference;

FIG. 5 is a flowchart showing steps for estimating the fractional fat content of an object of interest according to the method of FIG. 3;

FIG. 6 is a flowchart showing steps for grading an object of interest according to the method of FIG. 3;

FIG. 7 is a flowchart showing another embodiment of steps for estimating the fractional fat content of an object of interest according to the method of FIG. 3;

FIG. 8 is a flowchart of an image correction method;

FIG. 9 is another exemplary region of interest containing an object of interest and a reference;

FIG. 10 is a flowchart of another image correction method;

FIG. 11 shows an ultrasound transducer array beam having an elevational width smaller and larger than a blood vessel;

FIG. 12 is a graph showing exemplary bipolar signals obtained by the imaging system of FIG. 1; and

FIG. 13 is a flowchart showing another embodiment of steps for estimating the fractional fat content of an object of interest according to the method of FIG. 3.

### DETAILED DESCRIPTION OF THE EMBODIMENTS

In the following, a method and system for estimating fractional fat content of an object of interest will be described. Generally, the method comprises obtaining thermoacoustic data of a region of interest containing an object of interest and a reference. Fractional fat content of the object of interest is estimated using the thermoacoustic data and at least one parameter of the reference. The object of interest is graded using the estimated fractional fat content thereof.

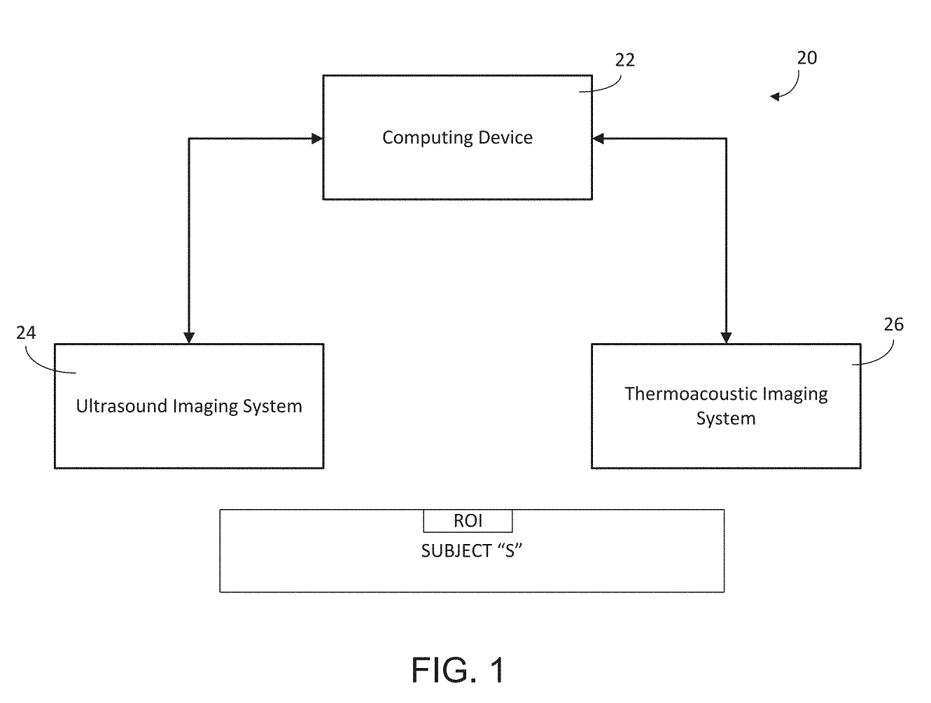

Turning now to FIG. 1, an imaging system is shown and is generally identified by reference numeral 20. As can be seen, in this embodiment the imaging system 20 comprises a computing device 22 communicatively coupled to an ultrasound imaging system 24 and a thermoacoustic imaging system 26. The ultrasound imaging system 24 and thermoacoustic imaging system 26 are configured to obtain ultrasound image data and thermoacoustic data, respectively, of a region of interest ROI associated with a subject S.

The computing device 22 in this embodiment is a personal computer or other suitable processing device comprising, for example, a processing unit comprising one or more processors, system memory (volatile and/or non-volatile memory), other non-removeable or removable memory (e.g., a hard disk drive, RAM, ROM, EEPROM, CD-ROM, DVD, flash memory, etc.) and a system bus coupling the various computer components to the processing unit. The computing device 22 may also comprise networking capabilities using Ethernet, and/or other suitable network format, to enable connection to shared or remote drives, one or more networked computers, or other networked devices. One or more input devices, such as a mouse and a keyboard (not shown) are coupled to the computing device 22 for receiving user input. A display device (not shown), such as a computer screen or monitor, is coupled to the computing device 22 for displaying one or more generated images that are based on ultrasound image data received from the ultrasound imaging system 24 and/or the thermoacoustic data received from thermoacoustic imaging system 26.

The ultrasound imaging system 24 comprises one or more ultrasound transducer arrays (not shown) configured to emit sound waves into the region of interest ROI of the subject. In this embodiment, the one or more ultrasound transducer arrays are disconnectable from the ultrasound imaging system 24. The sound waves directed into the region of interest ROI of the subject echo off tissue within the region of interest ROI, with different tissues reflecting varying degrees of sound. These echoes are received by the one or more ultrasound transducer arrays and are processed by the ultrasound imaging system 24 before being communicated as ultrasound image data to the computing device 22 for further processing and for presentation and interpretation by an operator. In this embodiment the ultrasound imaging system 24 utilizes B-mode ultrasound imaging techniques assuming a nominal speed of sound of 1,540 m/s. As ultrasound imaging systems are known in the art, further specifics of the ultrasound imaging system 24 will not be described further herein.

The thermoacoustic imaging system 26 comprises a radio-frequency (RF) source (not shown) configured to generate short pulses of RF electromagnetic radiation that are directed into the region of interest ROI of the subject to deliver energy to tissue within the region of interest ROI of the subject. The energy delivered to the tissue induces acoustic pressure waves that are detected by the thermoacoustic imaging system 26 using one or more ultrasound transducer arrays (not shown). In this embodiment, the thermoacoustic imaging system 26 makes use of the one or more ultrasound transducer arrays of the ultrasound imaging system 26 by disconnecting the one or more ultrasound transducer arrays of the ultrasound imaging system 24 and connecting them to the thermoacoustic imaging system 26 and as such, coordinate mapping between ultrasound transducer arrays is not required. In this embodiment, the RF source has a frequency between about 10 Mhz and 100 Ghz and has a pulse duration between about 0.1 nanoseconds and 10 microseconds. Acoustic pressure waves detected by the one or more ultrasound transducer arrays are processed and communicated as thermoacoustic data to the computing device 22 for further processing and for presentation and interpretation by an operator. As thermoacoustic imaging systems are known in the art, further specifics of the thermoacoustic imaging system 26 will not be described further herein.

Thermoacoustic imaging can be used to contrast between fat and fatty tissues due to their lower electrical conductivity and permittivity in RF compared to other water and ion-rich soft tissues. Fat and fatty tissues also have a lower absorption coefficient compared to soft tissues like muscle. As such, obtaining thermoacoustic data of both fatty and soft tissues results in a bipolar signal at a boundary between the fatty tissue and the soft tissue. The strength of the bipolar signal depends on the relative absorption properties of the fatty tissue and the soft tissue. Further details can be found in the following references: “Scanning thermoacoustic tomography in biological tissue” authored by Ku et al., Med. Phys., vol. 27, no. 5, pp. 1195-202, May 2000; “Microwave-induced thermoacoustic imaging model for potential breast cancer detection” authored by Wang et al., IEEE Trans. Biomed. Eng., vol. 59, no. 10, pp. 2782-01, October 2012; and “IT′IS Database for thermal and electromagnetic parameters of biological tissues” authored by Hasgall et al. Version 3.0, September 2015.

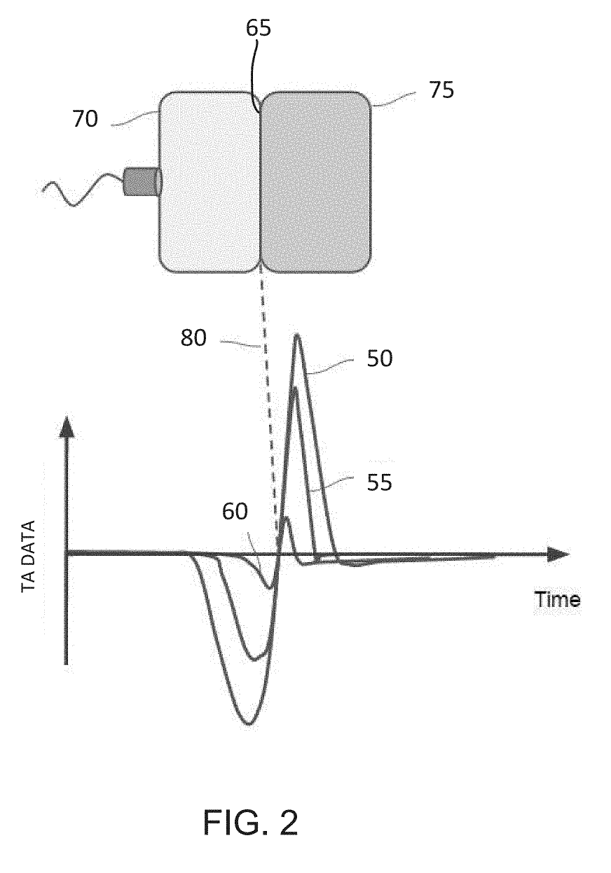

Exemplary bipolar signals 50, 55 and 60 are shown in FIG. 2. The bipolar signals 50, 55 and 60 represent thermoacoustic data obtained at a boundary 65 between fatty tissue 70 and lean tissue 75. The dashed line 80 indicates a time point corresponding to the boundary 65. The peak-to-peak value of each bipolar signal 50, 55 and 60 is proportional to a difference in absorption coefficient between the fatty tissue 70 and lean tissue 75. As such, thermoacoustic data associated with a boundary between tissue having no fat (such as a kidney) and tissue having a high fraction fat content (such as a fatty liver) results in bipolar signal 50. Thermoacoustic data associated with a boundary between tissue having no fat (such as a kidney) and tissue having a medium fractional fat content (such as an unhealthy liver) results in bipolar signal 55. Thermoacoustic data associated with a boundary between tissue having no fat (such as a kidney) and tissue having a low fractional fat content (such as a healthy liver) results in bipolar signal 60.

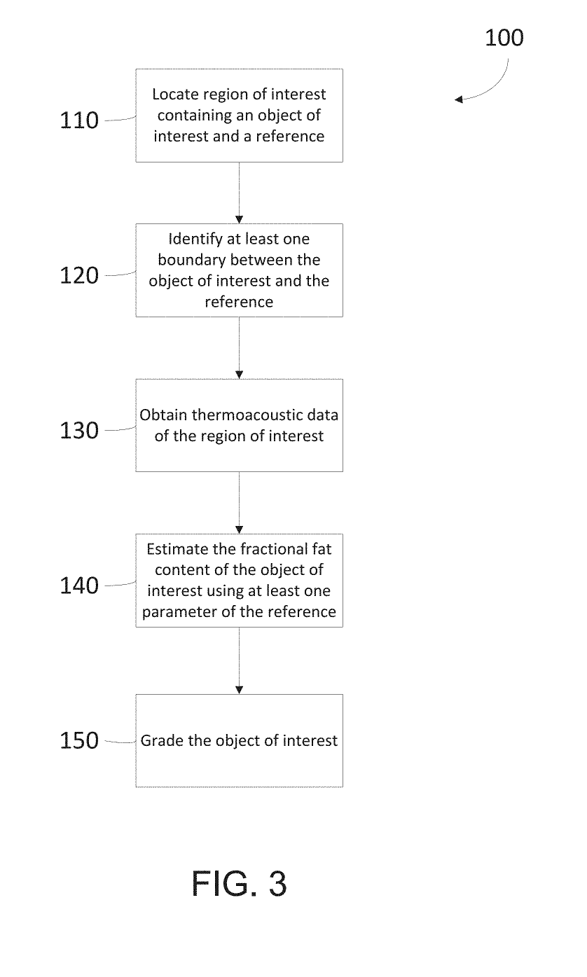

The imaging system 20 exploits the characteristic bipolar signals in obtained thermoacoustic data to estimate fractional fat content of an object of interest. In this embodiment, the imaging system 20 performs a method 100 for grading an object of interest based on the fractional fat content thereof, as will now be described with reference to FIG. 3.

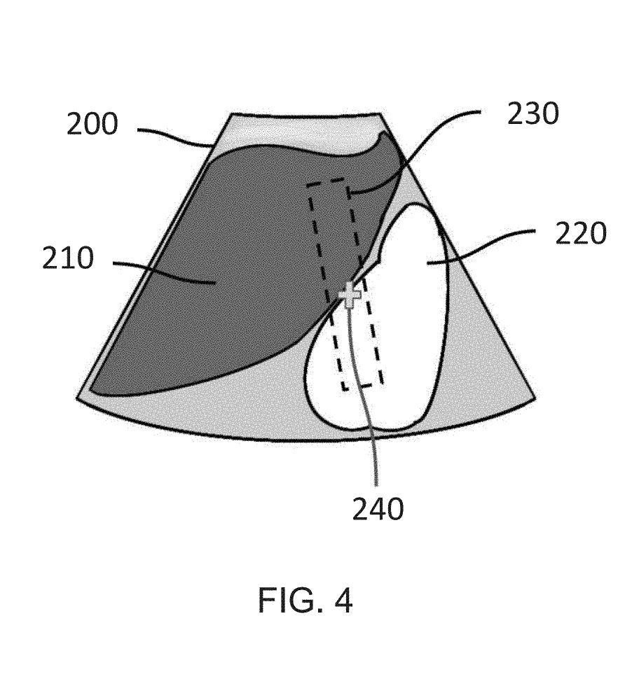

During the method, a region of interest is initially located within a subject's body that contains an object of interest and a reference (step 110). In this embodiment, the region of interest is located using the ultrasound imaging system 24. Specifically, ultrasound image data obtained by the ultrasound imaging system 24 is communicated to the computing device 22. The ultrasound image data is processed by the computing device 22 and a reconstructed ultrasound image is presented on the display device. The operator moves the one or more ultrasound transducer arrays on the subject's body until the region of interest is located. When locating the region of interest, the computing device 22 overlays information associated with the angle of the axial axis (or ultrasound transducer array beam axis) of the one or more transducer arrays overtop of the reconstructed ultrasound image on the display device. The information is used to provide feedback to the operator to ensure the axial axis of the one or more transducer arrays are generally perpendicular to a boundary between the object of interest and the reference. An exemplary region of interest 200 containing an object of interest 210 and a reference 220 is shown in FIG. 4. In this embodiment, the object of interest 210 is the subject's liver 210 and the reference is the subject's kidneys 220.

At least one boundary between the object of interest and the reference is then identified (step 120). In this embodiment, the at least one boundary is identified by the operator using an input device such as a mouse coupled to the computing device 22. Specifically, the operator draws a box that includes at least a portion of the object of interest, at least a portion of the reference and the boundary between the object of interest and the reference. The computing device 22 provides feedback to the user via the display device to indicate the approximate angle between the box and the boundary to ensure the box is generally perpendicular to the boundary.

An exemplary box 230 is shown in FIG. 4. As can be seen, the box 230 includes a portion of the liver 210, a portion of the kidney 220 and a boundary 240 between the liver 210 and the kidney 220. The boundary 240 is selected at a particular location wherein the liver 210 and the kidney 220 are in close relation to one another.

Thermoacoustic data of the region of interest is then obtained using the thermoacoustic imaging system 26 (step 130). As will be appreciated, an ultrasound image grid is defined by size, its position relative to the region of interest and a unit-cell (voxel) size. The ultrasound image grid and position are defined such that the boundary is enclosed within the grid. From the ultrasound image grid, a thermoacoustic measurement grid is constructed to ensure registration of the thermoacoustic image location to the ultrasound image coordinates. In this embodiment, since the thermoacoustic data is obtained using one of the ultrasound transducer arrays used for obtaining the ultrasound image data and, the thermoacoustic measurement grid is easily constructed. Specifically, the thermoacoustic measurement grid is equal to the ultrasound image grid.

Using the thermoacoustic data, the fractional fat content of the object of interest is estimated using at least one known parameter of the reference (step 140). In this embodiment, the absorption coefficient of the reference is known based on previously obtained data. Using the known absorption coefficient of the reference, the absorption coefficient of the object of interest is estimated. Using the estimated absorption coefficient of the object of interest, the fractional fat content of the object is estimated.

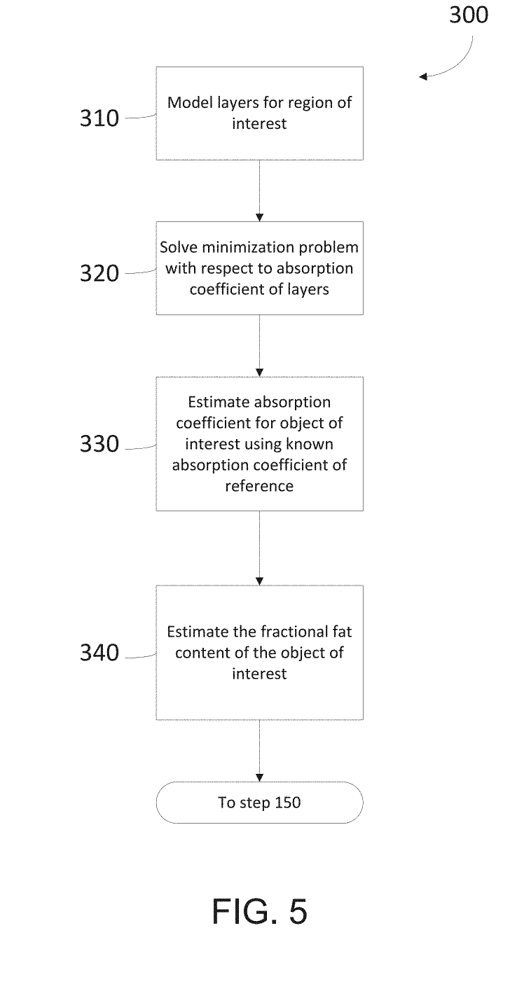

An exemplary method 300 of estimating the fractional fat content of the object of interest at step 140 is shown in FIG. 5. During the method, the region of interest is modeled in layers (step 310). Generally, the region of interest is constructed as layers with known non-fat tissues and unknown fat tissues. As such, it is assumed that these layers are homogeneous. As will be appreciated, the thermoacoustic pressure produced by a heat source H(r, t) obeys the following equation:

$${∇}^{2} p ( r , t ) - \frac{1}{{c}^{2}} \frac{{\partial }^{2}}{\partial {t}^{2}} p ( r , t ) = - \frac{β}{{C}_{p}} \frac{\partial }{\partial t} H ( r , t ) \qquad [ 1 ]$$

where β is the isobaric volume expansion coefficient, c is the sound speed and Cₚ is the specific heat capacity. Setting the position of the ultrasound transducer array as the origin of the coordinates, the forward problem at transducer position and time t is derived be solving equation 2:

$$p ( t ) = \frac{β}{4 π {C}_{p}} \int \int \int \int \frac{\partial r}{r} \frac{\partial H ( r , {t}^{′} )}{\partial {t}^{′}} {\vert}_{{t}^{′} = t - \frac{r}{c}} \qquad [ 2 ]$$

It is assumed that the heat source has a separable form, and thus:

H(r,t)=A(r)I(t) [3]

where A(r) is the spatial distribution of energy absorption and I(t) is the temporal irradiation function. Since the ultrasound transducer array has a finite bandwidth, the recorded thermoacoustic measurements p_{d}(t) are the convolution of induced pressure p(t) and the impulse response of the ultrasound transducer array h(t) as set out in equation 4:

P_{d}(t)=p(t)*h(t) [4]

where * denotes a one-dimensional (1D) temporal convolution.

As will be appreciated, for conventional thermoacoustic imaging, the goal is to recover the absorption coefficient A(r) by inverting the forward problem. The thermoacoustic pressure for a layer with thickness d under short-pulse radiation can be expressed by equation 5:

$$p ( t ) = {μ}_{a} {I}_{0} \frac{β {c}^{2}}{{C}_{p}} u ( t , {d}_{0} ) \qquad [ 5 ]$$

where μₐ is the absorption property of the layer, I₀ is the radiation intensity at the layer, d₀ is the distance from the ultrasound transducer array to the layer, and u(t, d₀) is a function whose value is equal to one (1) when d₀≦ct≦d₀+d and equal to zero (0) otherwise. Because of the impulse response characteristic of the ultrasound transducer array, the recorded thermoacoustic measurements exhibit bipolar signals at edges of the layer.

Since tissue with a higher fractional fat content will have different dielectric and thermal properties than lean (no fat content) tissue, the fractional fat content of a tissue nₜᵢₛₛᵤₑ can be deduced from thermoacoustic measurements. To model the region of interest in layers, the fractional fat content is deduced by assuming that the region of interest can be modeled by piecewise constant layers of tissues having different absorption coefficients as outlined in equation 6:

A(r)=Σᵢxᵢlᵢ(r) [6]

where x=[. . . xᵢ . . . ]^{T} is the layer value vector to be estimated, and l₁(r) is the i^{th} layer, which in this embodiment is modeled as a rectangular block.

Using the forward models outlined by equations 2 and 4 and the object model outlined by equation 6, equation 7 is constructed:

Ψ(x)=∥y −Bx∥_{W}⁸ [7]

where y is the measurement vector, B=[. . . Flᵢ(r) . . . ], W is a weighting matrix, and F is the forward model for the thermoacoustic imaging system 26. The forward model F is outlined in the reference entitled “A constrained variable projection reconstruction method for photoacoustic computed tomography without accurate knowledge of transducer responses,” authored by Sheng et al., IEEE Trans. Med. Imag., vol. 34, no. 12, pp. 2443-58, December 2015. Matrix B provides basis functions with characteristic bipolar signals at the boundaries of the layers. The weighting matrix W may be used for various purposes including assessing the objective function only near the boundaries of the layers. In this embodiment, non-negativity constraint is enforced to reduce the search space to physically meaningful solutions. However, as will be appreciated, in other embodiments additional components may also be incorporated such as for example transducer weightings, a regularizer or a constrain, if some a priori information about the object is given.

An estimation is obtained by solving the given non-negative least squares minimization problem which provides the relative absorption properties of the layers (step 320). Using the known absorption coefficient of the reference, the absorption coefficient of the object of interest is estimated (step 330).

Using the estimated absorption coefficient of the object of interest, the fractional fat content of the object of interest is estimated (step 340). In this embodiment, the fractional fat content of the object of interest is estimated by comparing the absorption coefficient of the object of interest estimated in step 320 to tabulated absorption coefficients of tissues with different fat contents. In this embodiment, the tabulated absorption coefficients are obtained as outlined in “The dielectric properties of biological tissues: II. Measurements in the frequency range 10 Hz to 20 GHz,” authored by Gabriel et al., Med. Phys., vol. 41, no.

11, pp. 2251-69, November 1996.

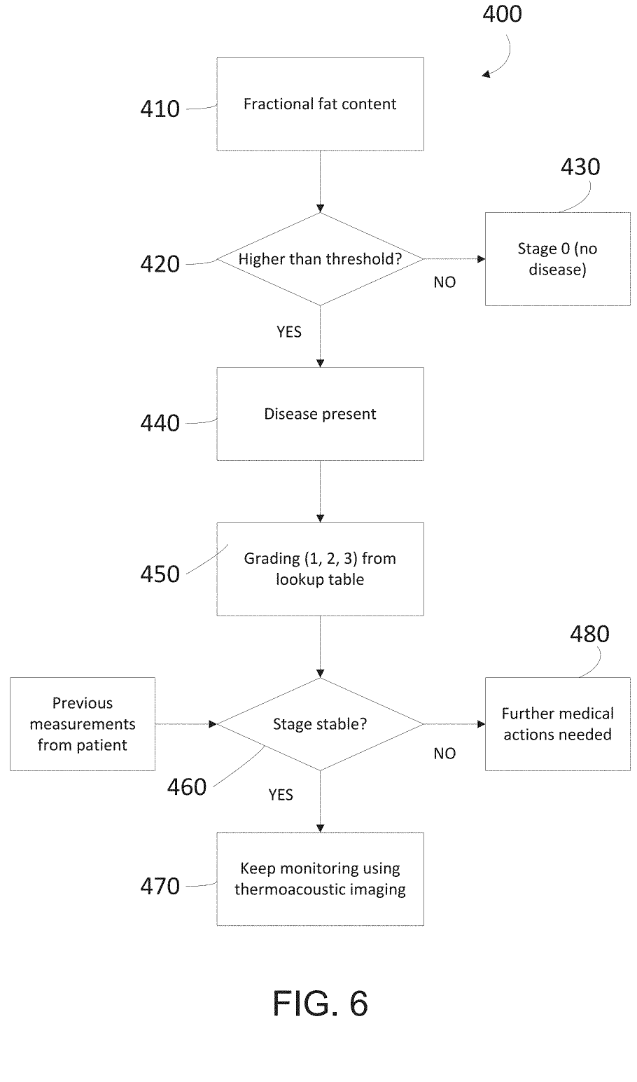

Turning back to FIG. 3, the object of interest is graded using the estimated fractional fat content (step 160). In this embodiment, the object of interest is graded according to a method 400 shown in FIG. 6. During the method, the estimated fractional fat content (step 410) is compared to a threshold (step 420). In this embodiment, the threshold is for fatty liver disease and is set at a fractional fat content of 5%. Specifics of the threshold for fatty liver disease are outlined in “Magnetic resonance imaging and liver histology as biomarkers of hepatic steatosis in children with non-alcoholic fatty liver disease,” authored by Schwimmer, Hepatology, vol. 61, pp. 1887-1895, 2015.

If the estimated fractional fat content is less than the threshold, it is determined that the subject does not have a disease and thus the object of interest is graded as a zero (0) (step 430). If the estimated fractional fat content is higher than the threshold, it is determined that a disease such as steatosis is present (step 440). The object of interest is in turn graded as a one (1), two (2) or three (3) by comparing the estimated fractional fat content to known tabulated values (step 450). In this embodiment, the known tabulated values are outlined in “Non-alcoholic steatohepatitis: A proposal for grading and staging the histological lesions,” authored by Brunt et al., Am. J. Gastroenterol., vol. 94, no. 9, pp. 2467-2474, September 1999.

Specifically, in this embodiment, the object of interest is graded as a one (1) if the estimated fractional fat content is between 5% and 33%. The object of interest is graded as a two (2) if the estimated fractional fat content is between 34% and 66%. The object of interest is graded as a three (3) if the estimated fractional fat content is greater than 66%.

The grade of the object of interest is then compared to previous grades obtained for the subject (if available) (step 460). If the grade of the object of interest has not changed, the object of interest is deemed stable and the subject is released (step 470). If the grade of the object of interest has changed, further medical actions are deemed to be required (step 480).

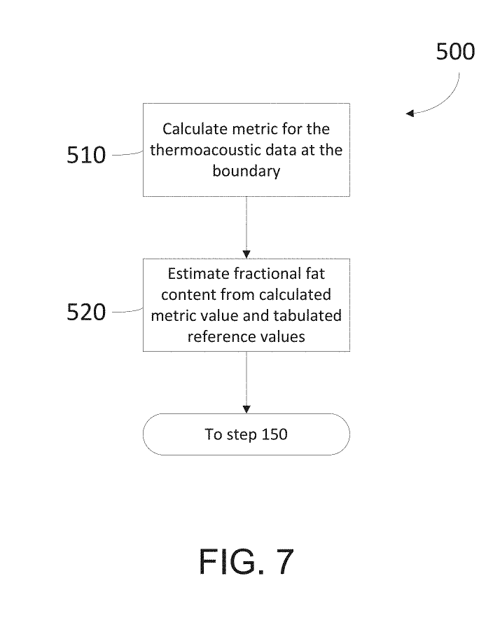

Although in embodiments described above the fractional fat content of the object of interest is estimated by determining an absorption coefficient of the object of interest, the fractional fat content of the object of interest may be determined in other ways. Turning to FIG. 7, another method for estimating the fractional fat content of the object of interest is shown and is generally identified with reference numeral 500. In this embodiment, previously defined equation 5 is used as a model to infer fractional fat content from the thermoacoustic data obtained in step 130. As will be appreciated, near the boundary, the transition from one tissue to another is approximated as a layer whose amplitude is proportional to the difference between thermoacoustic data of two adjacent tissues. As such, the strength of the bipolar signal at the boundary represents the absorption property difference between the two tissues. Further, the phase of measurement at the boundary indicates which tissue has a higher (or lower) absorption coefficient.

Using equation 5, the thermoacoustic data signal strength can be expressed as:

$$Sig ∝ \frac{{μ}_{a}}{{C}_{p}} \qquad [ 8 ]$$

In addition to the fat-fraction-dependent absorption coefficient μₐ, the tissue heat capacity Cₚ is also assumed to be dependent on fractional fat content. As such, the magnitude of the thermoacoustic data Sig_{boundary} at the boundary of two tissues can be expressed as:

$${Sig}_{boundary} ∝ \frac{{μ}_{a , 1}}{{C}_{p , 1}} - \frac{{μ}_{a , 2}}{{C}_{p , 2}} \qquad [ 9 ]$$

where μ_{a,1} and μ_{a,2} are absorption coefficients of two neighbouring tissues, and C_{p,1} and C_{p,2} are specific heat capacities of the two neighbouring tissues.

A metric for the strength of the thermoacoustic data at the boundary of the object of interest and the reference is calculated (step 510). In this embodiment, the thermoacoustic data represents a bimodal signal and the metric is the distance between two peaks of the bimodal signal. The metric is compared to tabulated reference values to estimate the fractional fat content of the object of interest (step 520). In this embodiment, the tabulated data is obtained by taking measurements of thermoacoustic data of various samples of tissue-mimicking phantoms having various fractional fat contents as outlined in “Spectroscopic thermoacoustic imaging of water and fat composition,” authored by Bauer, Appl. Phys. Lett., vol. 101, no. 3, July 2012.

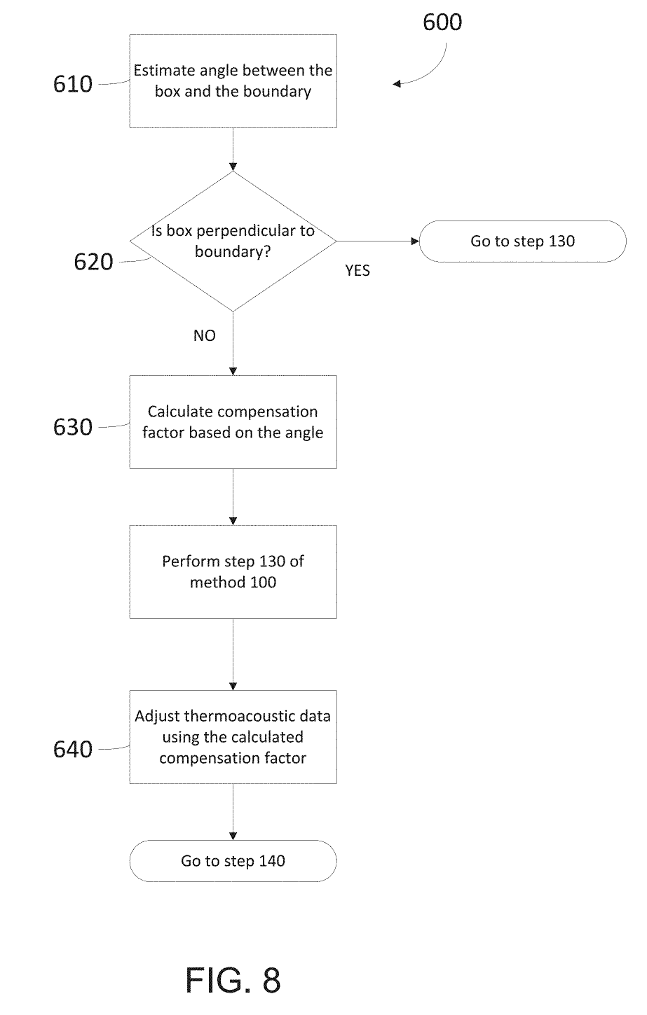

Although in embodiments described above the computing device 22 provides feedback to the user via the display device to indicate the approximate angle between the box and the boundary to ensure the box is generally perpendicular to the boundary, those skilled in the art will appreciate that additions or alternatives are available. For example, the thermoacoustic data may be adjusted to correct for embodiments where the box is not perpendicular to the boundary. In this embodiment, an image correction method 600 may be used with method 200. Method 600 is shown in FIG. 8. In this embodiment, the computing device 22 estimates the angle between the box and the boundary (step 610). If the box is generally perpendicular to the boundary, then method 100 is performed as outlined in FIG. 3 (step 620). If the box is not generally perpendicular to the boundary, then a compensation factor is calculated based on the angle of the box with respect to the boundary (step 630). Thermoacoustic data obtained in step 130 of method 100 is adjusted using the calculated compensation factor (step 640). Method 100 then continues to step 140 as shown in FIG. 3.

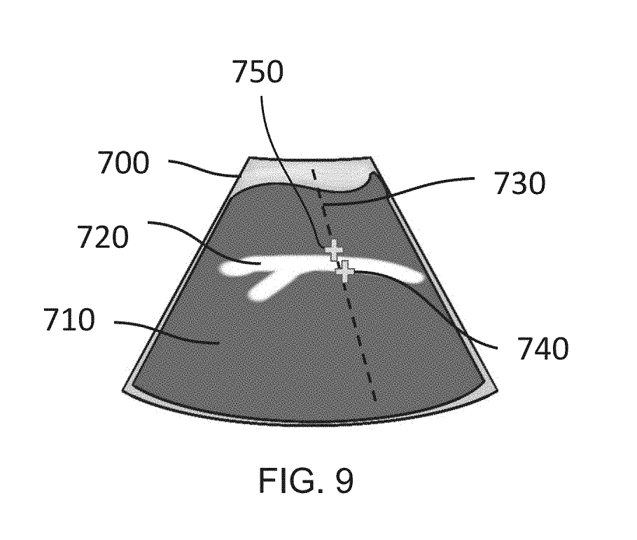

Although in embodiments described above, the reference is described as being a kidney located adjacent to the liver, those skilled in the art will appreciate that other types of non-fatty tissue may be used. For example, in another embodiment one or more blood vessels may be used as the reference. In this embodiment, the reconstructed ultrasound image displayed on the display device may be used by the operator to identify and select the one or more blood vessels. An example is shown in FIG. 9. As can be seen, a region of interest 700 includes the object of interest 710 which in this embodiment is the liver. The region of interest 700 also includes a reference 720 which in this embodiment is a blood vessel, specifically the portal vein. Of course, other blood vessels may be used. During step 120 of method 100, the at least one boundary is identified by the operator by drawing a line 730 that intersect boundaries 740, 750 between the reference 720 and the object of interest 710.

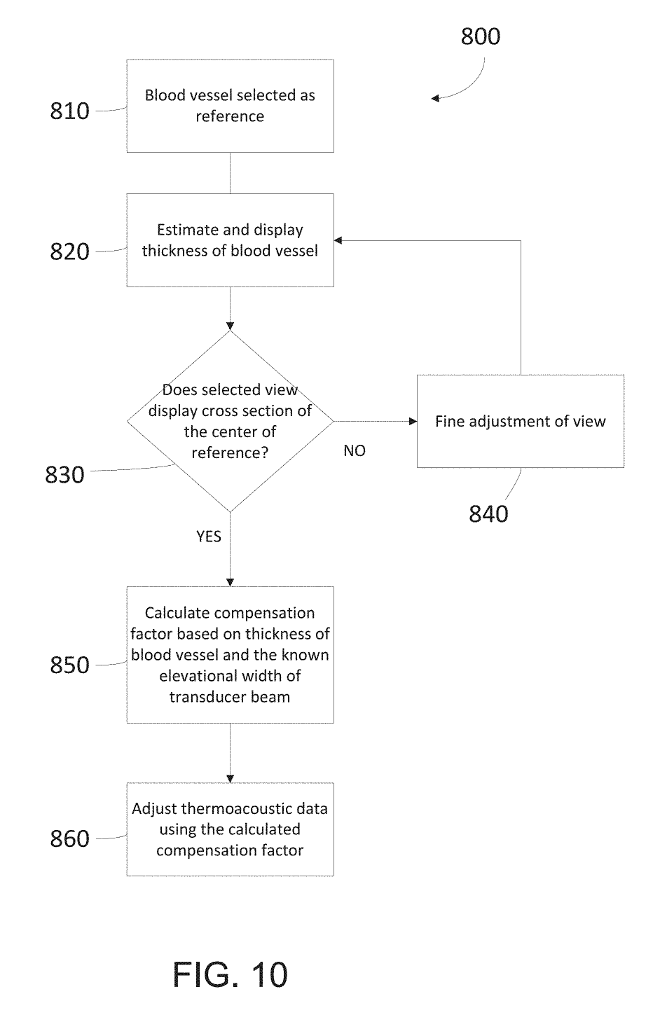

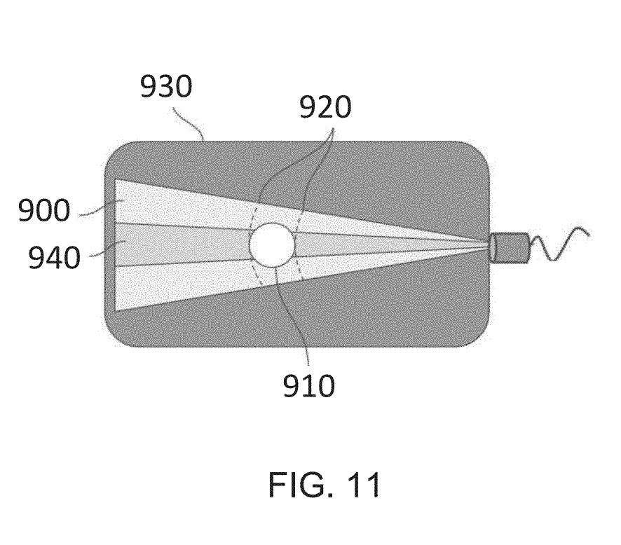

Since blood vessels (in this embodiment the portal vein) are generally thin, an image correction method 800 may be used with method 200. Method 800 is shown in FIG. 10. Once a blood vessel has been identified as the reference by the operator (step 810), the thickness of the blood vessel is estimated and displayed on the display device (step 820). In this embodiment, the thickness of the blood vessel is estimated using segmentation methods executed by the computing device 22. A check is performed to determine if the current view of the display device shows a cross-section of the blood vessel (step 830). If the current view does not show the cross-section of the blood vessel, the view of the display device is adjusted (step 840) until the current view shows the cross-section. A compensation factor is calculated based on the estimated thickness of the blood vessel and the known elevational width of the ultrasound transducer array beam (step 850). As shown in FIG. 11, the elevational width of the ultrasound transducer array beam 900 is greater than a thickness of reference blood vessel 910. As such, the thermoacoustic data associated with the boundaries 920 between an object of interest 930 and reference blood vessel 910 is affected by a ratio of the volume of the object of interest and the volume of the reference within the ultrasound transducer array beam. The elevational width of ultrasound transducer beam 940 is less than a thickness of reference blood vessel 910. Similarly, the thermoacoustic data associated with the boundaries 920 between the object of interest 930 and reference blood vessel 910 is affected by a ratio of the volume of the object of interest and the volume of the reference within the ultrasound transducer array beam. Accordingly, the compensation factor is calculated. Thermoacoustic data obtained in step 130 of method 100 is adjusted using the calculated compensation factor (step 860). Method 100 then continues to step 140 as shown in FIG. 3.

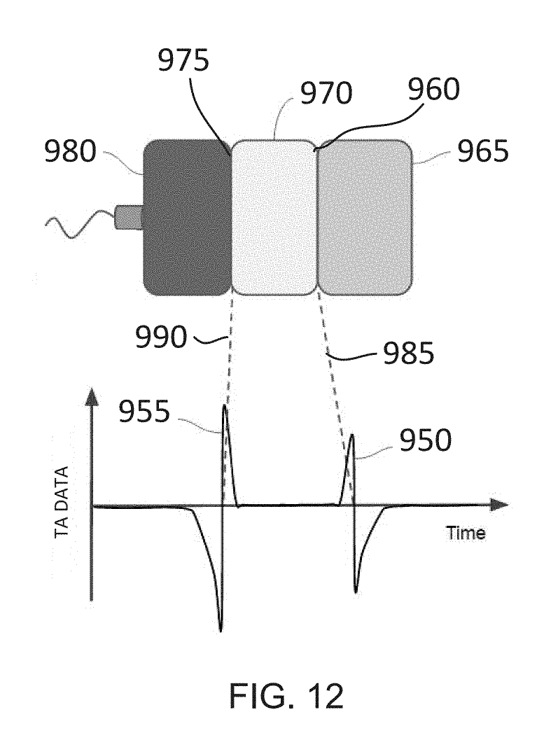

Although in embodiments described above the boundary is selected at a location where the object of interest and the reference are in close relation to one another, those skilled in the art will appreciate that alternatives are available. For example, in another embodiment an intermediate structure may be in between the reference and the object of interest. Exemplary bipolar signals 950 and 955 of this embodiment are shown in FIG. 12. The bipolar signals 950 and 955 represent thermoacoustic data obtained at a boundary 960 between reference 965 and intermediate structure 970 and a boundary 975 between intermediate structure 970 and object of interest 980, respectively. The dashed line 985 indicates a time point corresponding to the boundary 960 and dashed line 990 indicates a time point corresponding to the boundary 975.

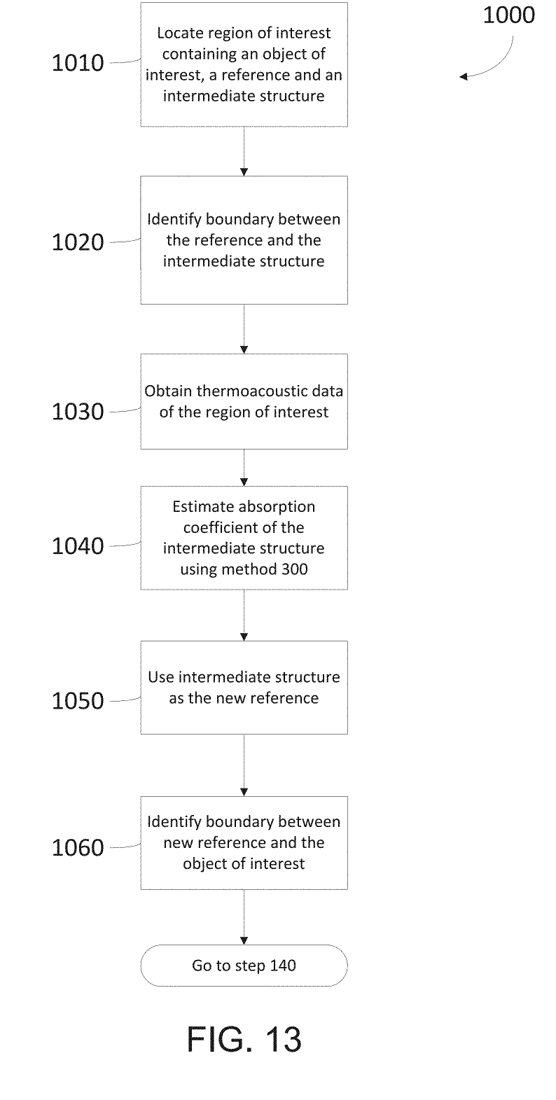

In this embodiment, steps 110 to 130 of method 100 are modified as shown in method 1000 of FIG. 13. A region of interest is located similar to step 110 of method 100 (step 1010). In this embodiment, the region of interest contains an object of interest, a reference and an intermediate structure in between the reference and the object of interest. A boundary between the reference and the intermediate structure is identified similar to step 120 of method 100 (step 1020). Thermoacoustic data is obtained of the region of interest (step 1030). The absorption coefficient of the intermediate structure is estimated using method 300 (step 1040). Now that the absorption coefficient of the intermediate structure is known, the intermediate structure is used as a new reference (step 1050). A boundary between the new reference and the object of interest is identified similar to step 120 of method 100 (step 1060). The method then continues to step 140 wherein the fractional fat content of the object of interest is estimated using the absorption coefficient of the new reference.

In another embodiment, more than one intermediate structure may be between the reference and the object of interest. In this embodiment, steps 1010 to 1060 of method 1000 are repeated until the absorption coefficient of an intermediate structure adjacent to the object of interest is estimated. The method then continues to step 140 wherein the fractional fat content of the object of interest is estimated using the absorption coefficient of the intermediate structure adjacent to the object of interest.

Although in embodiments described above the reference is described as being selected by the operator, those skilled in the art will appreciate that alternatives are available. For example, in another embodiment the reference may be automatically defined using an algorithm performed by the computing device 22 based on known geometry and/or known ultrasound properties of particular types of tissue within the region of interest.

Further, the boundary between the reference and the object of interest may be automatically defined using algorithms based on ultrasound segmentation or thermoacoustic data analysis. As will be appreciated, both operator-defined and automatic methods may be combined.

Although in embodiments above the one or more ultrasound transducer arrays are described as being disconnectable from the ultrasound imaging system 24 and reconnectable to the thermoacoustic imaging system 26, those skilled in the art will appreciate that alternatives are possible. For example, the ultrasound imaging system 24 and the thermoacoustic imaging system 26 may have their own respective one or more transducer arrays. In another embodiment, the one or more ultrasound transducer arrays may be connected to a hub which itself is connected to the ultrasound imaging system and the thermoacoustic imaging system. In this embodiment, the hub may be controlled by the computing device 22 or by other input to switch operation between the ultrasound imaging system and the thermoacoustic imaging system and vice versa.

Although in embodiments described above a metric used with method 500 is described as being the difference between two peaks of a bimodal signal, those skilled in the art will appreciate that the metric may be a simple peak (maximum), a p-norm, area under the bimodal signal, etc.

As will be appreciated, embodiments of image processing described above can be performed on ultrasound and thermoacoustic images in real-time or off-line using images stored in memory.

Although in embodiments described above the region of interest is described as being modeled in layers, alternatives are available. For example, in another embodiment the region of interest may be modeled in layers with more complex functions, such as for example higher order polynomials. In other embodiments, the region of interest may be modeled using interpolation methods such as for example cubic spline interpolation.

Although the thermoacoustic imaging system is described as comprising an RF source configured to generate short pulses of RF electromagnetic radiation, those skilled in the art will appreciate that in other embodiments the thermoacoustic imaging system may comprise a visible light source or an infrared radiation source with a wavelength between 400 nm and 10 μm and a pulse duration between 10 picoseconds and 10 microseconds.

Although in embodiments described above the thermoacoustic imaging system and the ultrasound imaging system are described as using one or more ultrasound transducer arrays, those skilled in the art will appreciate that the alternatives are available. For example, a single transducer element, an ultrasound transducer array having a linear or curved one-dimensional array, or a two-dimensional ultrasound transducer array may be used. In addition, a gel-like material or water capsule may be used to interface the one or more ultrasound transducer arrays with the region of interest.

Although in embodiments described above, the fractional fat content of the object of interest is estimated using thermoacoustic data obtained of a single region of interest, those skilled in the art will appreciate that multiple regions of interest may be analyzed and combined.

Although in embodiments described above blood vessels are described as being identified manually by an operator, those skilled in the art will appreciate that blood vessels may be identified in other ways. For example, in another embodiment automatic or semi-automatic algorithms may be used to identify one or more blood vessels. In other embodiments, Doppler imaging methods may be used to identify blood vessels.

Those skilled in the art will appreciate that the above-described ultrasound image data and thermoacoustic data may be one-dimensional, two-dimensional or three-dimensional. In embodiments, the ultrasound image data may be in a different dimension than the thermoacoustic data. For example, ultrasound image data may be two-dimensional and the thermoacoustic data may be one-dimensional. Further, different fields of view may be used.

In another embodiment, different types or models of transducer arrays may be used with the thermoacoustic and ultrasound imaging systems. In this embodiment, a transform may be used to map a thermoacoustic absorption image to the ultrasound image. In another embodiment, in the event that knowledge of transducer array geometry is not readily available, the thermoacoustic absorption image may be mapped to the ultrasound image using phantom reference points. In this embodiment, a transform may be used to map known phantom reference points from the thermoacoustic absorption image to the phantom reference points on the ultrasound image.

Although the ultrasound imaging system is described as using B-mode ultrasound imaging techniques, other techniques may be used such as for example power Doppler images, continuous wave Doppler images, etc.

Those skilled in the art will appreciate that other objects of interest may be evaluated and other references may be used such as for example the heart, kidney(s), lung, esophagus, thymus, breast, prostate, brain, muscle, nervous tissue, epithelial tissue, bladder, gallbladder, intestine, liver, pancreas, spleen, stomach, testes, ovaries, uterus, skin and adipose tissues.

Although in embodiments described above thermoacoustic data is obtained of the region of interest, those skilled in the art will appreciate that thermoacoustic data may be obtained for an area larger than the region of interest.

Using the foregoing specification, the invention may be implemented as a machine, process or article of manufacture by using standard programming and/or engineering techniques to produce programming software, firmware, hardware or any combination thereof.

Any resulting program(s), having computer-readable instructions, may be stored within one or more computer-usable media such as memory devices or transmitting devices, thereby making a computer program product or article of manufacture according to the invention. As such, functionality may be imparted on a physical device as a computer program existent as instructions on any computer-readable medium such as on any memory device or in any transmitting device, that are to be executed by a processor.

Examples of memory devices include, hard disk drives, diskettes, optical disks, magnetic tape, semiconductor memories such as FLASH, RAM, ROM, PROMS, and the like. Examples of networks include, but are not limited to, the Internet, intranets, telephone/modem-based network communication, hard-wired/cabled communication network, cellular communication, radio wave communication, satellite communication, and other stationary or mobile network systems/communication links.

A machine embodying the invention may involve one or more processing systems including, for example, computer processing unit (CPU) or processor, memory/storage devices, communication links, communication/transmitting devices, servers, I/O devices, or any subcomponents or individual parts of one or more processing systems, including software, firmware, hardware, or any combination or subcombination thereof, which embody the invention as set forth in the claims.

Using the description provided herein, those skilled in the art will be readily able to combine software created as described with appropriate special purpose computer hardware to create a computer system and/or computer subcomponents embodying the invention, and to create a computer system and/or computer subcomponents for carrying out the method of the invention.

Although embodiments have been described above with reference to the accompanying drawings, those of skill in the art will appreciate that variations and modifications may be made without departing from the scope thereof as defined by the appended claims.

What is claimed is:

1. A method for estimating fractional fat content of an object of interest, the method comprising:
obtaining thermoacoustic data of a region of interest containing an object of interest and at least one reference; and
estimating fractional fat content of the object of interest using the thermoacoustic data and at least one parameter of the reference.

2. The method of claim 1 wherein the at least one parameter is an absorption coefficient of the at least one reference.

3. The method of claim 2 further comprising estimating an absorption coefficient of the object of interest using the absorption coefficient of the at least one reference.

4. The method of claim 1 further comprising:
identifying a boundary between the object of interest and the at least one reference.

5. The method of claim 1 further comprising calculating a metric at the boundary between the object of interest and the reference using the thermoacoustic data and estimating an absorption coefficient of the object of interest using the metric.

6. The method of claim 1 further comprising grading the object of interest using the estimated fractional fat content.

7. The method of claim 6 wherein the grading comprises comparing the calculated fractional fat content to a threshold value.

8. The method of claim 6 wherein the grading comprising comparing the calculated fractional fat content to a lookup table.

9. The method of claim 1 further comprising:
obtaining ultrasound image data of the region of interest; and
registering coordinate frames of the thermoacoustic data and ultrasound image data.

10. The method of claim 9 wherein the registering comprises mapping coordinates of the thermoacoustic data with coordinates of ultrasound image data.

11. The method of claim 9 further comprising:
locating the region of interest using the obtained ultrasound image data.

12. The method of claim 11 further comprising:
identifying the at least one reference using the obtained ultrasound image data.

13. The method of claim 5 wherein the object of interest is a liver and the grading comprises grading hepatic steatosis.

14. The method of claim 13 wherein the at least one reference is at least one of a blood vessel within the liver and a kidney adjacent to the liver.

15. A method for grading an object of interest, the method comprising:
obtaining thermoacoustic data of a region of interest containing an object of interest and at least one reference;
estimating fractional fat content of the object of interest using the thermoacoustic data and at least one parameter of the at least one reference; and
grading the object of interest using the estimated fractional fat content.

16. The method of claim 14 wherein the grading comprises comparing the calculated fractional fat content to a threshold value.

17. The method of claim 14 wherein the grading comprising comparing the calculated fractional fat content to a lookup table.

18. The method of claim 14 wherein the object of interest is a liver and the grading comprises grading hepatic steatosis.

19. The method of claim 17 wherein the at least one reference is at least one of a blood vessel within the liver and a kidney adjacent to the liver.

20. A system comprising:
a thermoacoustic imaging system;
an ultrasound imaging system; and
a processing unit configured to:
process ultrasound image data received from the ultrasound imaging system to generate and display an ultrasound image of a region of interest containing an object of interest and at least one reference; and
process thermoacoustic data of the region of interest containing the object of interest and the at least one reference received from the thermoacoustic imaging system to estimate fractional fat content of the object of interest using at least one parameter of the at least one reference.

21. The system of claim 20 wherein the processing unit is further configured to:
process data received from an input device to identify the at least one reference within the ultrasound image.

22. A non-transitory computer-readable medium having stored thereon a computer program comprising computer program code executable by a computer to perform a method comprising:
obtaining thermoacoustic data of a region of interest containing an object of interest and at least one reference; and
estimating fractional fat content of the object of interest using the thermoacoustic data and at least one parameter of the at least one reference.

23. The non-transitory computer-readable medium of claim 22, wherein the method further comprises:
grading the object of interest using the estimated fractional fat content.

24. The non-transitory computer-readable medium of claim 22, wherein the method further comprises:
locating the object of interest and the at least one reference using ultrasound image data; and
identifying the at least one reference based on data received from an input device.
